(19) United States
(12) Patent Application Publication
(10) Pub. No.: US 2015/0374590 A1
(43) Pub. Date: Dec. 31, 2015

(54) ARTIFICIAL TEETH

(71) Applicant: UNIVERSITY OF WASHINGTON THROUGH ITS CENTER FOR COMMERCIALIZATION, Seattle, WA (US)

(72) Inventors: Daniel C. CHAN, Seattle, WA (US); Mark A. GANTER, Seattle, WA (US); John WATAHA, Seattle, WA (US); Julie HEWETT, Seattle, WA (US); Brandon BROWMAN, Seattle, WA (US); Kwok-hung CHUNG, Seattle, WA (US)

(21) Appl. No.: 14/767,669

(22) PCT Filed: Mar. 19, 2014
(86) PCT No.: PCT/US2014/031231

§ 371 Date: Aug. 13, 2015

Related U.S. Application Data
(60) Provisional application No. 61/803,344, filed on Mar. 19, 2013.

(51) Int. Cl. A61K 6/09; A61B 19/00; A61K 6/00; A61C 13/09

(52) U.S. Cl. CPC A61K 6/09; A61C 13/09; A61B 19/54; A61K 6/0002; A61K 6/0073; A61K 6/0088; A61B 2019/5466

(57) ABSTRACT

Disclosed herein are artificial teeth that include an enamel portion comprising a thermoset resin and a radio-opaque filler material distributed within the thermoset resin.

### RELATED APPLICATIONS

This application claims priority to U.S. Provisional Patent Application 61/803,344 filed Mar. 19, 2013, incorporated by reference herein in its entirety.

### BACKGROUND

Plastic (polymer resin) teeth used in dental schools for pre-clinical training can be used to simulate dental procedures (such as cavity preparation and restorative exercises); they do not adequately mimic natural teeth. The bond strength of dental composite materials to plastic teeth is inferior to natural tooth structure. This inferiority is most likely because acid-etching with 37% phosphoric acid does not differentially etch the plastic teeth. Without this differential etch, micromechanical bonding to a roughened surface cannot occur. This inability to bond leads to an unrealistic and, therefore, inadequate pre-clinical training.

Furthermore, today's plastic teeth are much softer than natural teeth. This limits the students' full development of mature motor skills in cavity preparation exercises because the teeth cannot appropriately mimic natural tooth structure.

### SUMMARY OF THE INVENTION

The present invention provides an artificial tooth for dental practice treatments, comprising

(a) an enamel portion, wherein the enamel portion comprises a thermoset resin, and wherein the enamel portion comprises an outer surface of the tooth; and

(b) a radio-opaque filler material distributed within the thermoset resin.

In one embodiment, the artificial tooth, further comprises a dentin portion, wherein the dentin portion is at least partially within the enamel portion, and wherein the enamel portion and the dentin portion together comprise a crown, wherein the dentin portion comprises a thermoset resin in which a radio-opaque filler material is distributed. In another embodiment, the thermoset resin and the radio-opaque filler making up the dentin are the same as those in the enamel portion.

In a further embodiment, the ratio of thermoset resin to radio-opaque filler material in the enamel portion is between about 1:1 to about 1.5:1 on a weight:volume basis. In a still further embodiment, the ratio of thermoset resin to radio-opaque filler material in the dentin portion is between about 1:1 to about 2:1 on a weight:volume basis. In another embodiment, the ratio of thermoset resin to radio-opaque filler material in the dentin portion is higher than the ratio of thermoset resin to radio-opaque filler material in the enamel portion.

In a further embodiment, the radio-opaque filler material has an average particle size of between about 15 microns and about 40 microns. In a still further embodiment, the enamel portion and the dentin portion have a porosity of between about 0% to about 5%. In another embodiment, the radio-opaque filler material comprises a silane coating.

In one embodiment, the thermoset resin comprises polyurethane; in another embodiment, the radio-opaque filler material comprises calcium carbonate. In various further embodiments, the artificial tooth may further comprise a root portion, wherein the crown is over the root portion, and/or a cavity.

In a second aspect, the invention provides methods for making an artificial tooth, comprising:

(a) distributing a radio-opaque filler material within a thermoset resin to provide a modified resin; and

(b) molding the modified resin into an artificial tooth.

### DESCRIPTION OF THE FIGURES

FIG. 1. Exemplary embodiment of an artificial tooth according to the present invention.

FIG. 2. Exemplary embodiment of an artificial tooth partially embedded in a dental arch model.

FIG. 3. Photograph of exemplary artificial teeth of the invention.

### DETAILED DESCRIPTION OF THE INVENTION

All references cited are herein incorporated by reference in their entirety. As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

As used herein, “about” means plus or minus 5% of the recited measurement.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the invention provides artificial teeth for dental practice treatments, comprising

(a) an enamel portion, wherein the enamel portion comprises a thermoset resin, and wherein the enamel portion comprises an outer surface of the tooth; and

(b) a radio-opaque filler material distributed within the thermoset resin.

As described in the examples that follow, the artificial teeth of the invention improve the hardness, differential etching and micromechanical bonding compared to existing dental composite materials, thereby providing an experience more relevant to the surgical preparation of natural teeth. The bond strength of dental composite materials to current plastic teeth is inferior to the bond strength to the natural tooth structure. This inferiority is most likely because acid-etching with 37% phosphoric acid does not differentially etch the plastic teeth. Without this differential etch, micromechanical bonding to a roughened surface cannot occur. This inability to bond leads to an unrealistic and, therefore, inadequate pre-clinical training. Furthermore, today's plastic teeth are much softer than natural teeth. This limits the students' full development of mature motor skills in cavity preparation exercises because the teeth cannot appropriately mimic natural tooth structure.

The present invention, by distributing the radio-opaque filler within the thermoset resin enamel potion allows differential etching; thus micromechanical bonding of dental composite materials to the artificial tooth is improved. In addition, the filler increases the tooth hardness, thereby providing an experience more relevant to the surgical preparation of natural teeth.

The artificial teeth of the present invention may be sized and shaped as appropriate for any intended use. In one embodiment, the teeth are formed as a block, with the thermoset resin forming the exterior of the block (i.e., as an outer layer). In another embodiment, the teeth are sized and shaped to simulate a natural tooth. In any of these embodiments, the teeth may further be attached and/or partially embedded in a surface, such as a dental arch model, as may be appropriate for a given use.

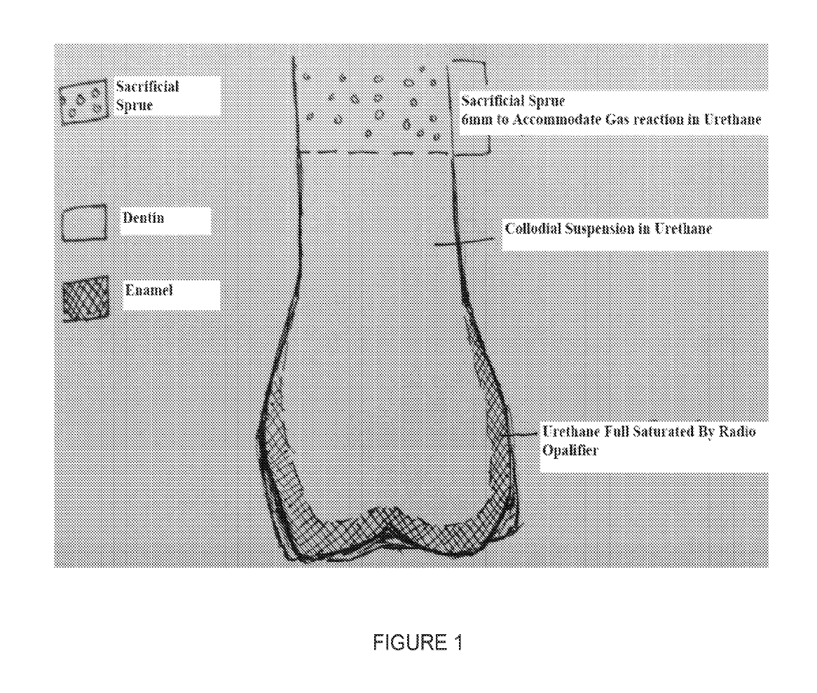

The enamel potion of the artificial tooth, which comprises an outer surface of at least a portion of the tooth, comprises or consists of a thermoset resin. FIG. 1 shows an exemplary embodiment of the tooth sized and shaped to simulate a natural tooth, wherein the enamel layer is present as an outer layer over only a portion of the tooth.

As used herein, a thermoset resin is a petrochemical in a soft solid or viscous state that changes irreversibly into an infusible, insoluble polymer network by curing. Curing can be induced by the action of heat or suitable radiation, or both. A cured thermosetting resin is called a thermoset. Exemplary thermoset resins included but are not limited to polyester resins, epoxy resins, cyanate ester resins, vinyl cure resins, ultraviolet cure resins, BMI's (bismaleimides), polybenzoxazine, polyimide, phenolic resin, polyesters, polyurethanes, and others known to those skilled in the art. In one specific embodiment, the thermoset resin comprises a polyurethane resin.

The filler comprises a radio-opaque material distributed within the thermoset resin. As used herein, the term “radio-opaque” means not transparent to X-rays or other forms of radiation. Exemplary radio-opaque fillers for use in the artificial teeth of the invention include, but are not limited to talc, silica, mica, calcium carbonate, barium sulfate, alumina, silica carbide, plastic monofilaments, carbon fiber, zirconia, borosilicate glass powder, radiopaque borosilicate powder, titanium dioxide, zinc oxide, and pigments. In one specific embodiment, the radio-opaque filler comprises calcium carbonate.

The filler may be distributed within the thermoset resin using any suitable technique; exemplary such techniques are provided in the samples that follow. The filler may be treated in any suitable way to facilitate its distribution within the thermoset resin. In one embodiment, the filler may be treated to improve binding to the thermoset resin, including but not limited to silane coating of the filler. The filler is distributed within the thermoset resin, in that it is relatively homogenously dispersed throughout the resin and not clumped or aggregated.

In one embodiment, the radio-opaque filler material has an average size of between about 400 mesh and about 600 mesh, or between about 15-40 microns in diameter. As will be understood by those of skill in the art, a preferred average particle size depends on a number of factors, including but not limited to the thermoset resin in which the filler particle is to be distributed, the viscosity of the thermoset resin, etc.

In one embodiment, the artificial tooth further comprises a dentin portion/layer, wherein the dentin portion is at least partially or completely within the enamel portion/layer (as in a natural tooth structure), wherein the enamel portion and the dentin portion together comprise a crown, wherein the dentin portion comprises a thermoset resin in which a radio-opaque filler material is distributed. In one embodiment, the teeth are formed as a block, with the enamel portion forming the exterior of the block (i.e., as an outer layer) and the dentin portion forming an internal layer. In another embodiment, the tooth is sized and shaped to simulate a natural tooth, wherein the enamel layer is present as an outer layer over the dentin portion only over a portion of the tooth (FIG. 1). The teeth may further be etched/shaped, for examples on the enamel at a top portion of the tooth (shown at bottom in FIG. 1) to simulate natural tooth structure.

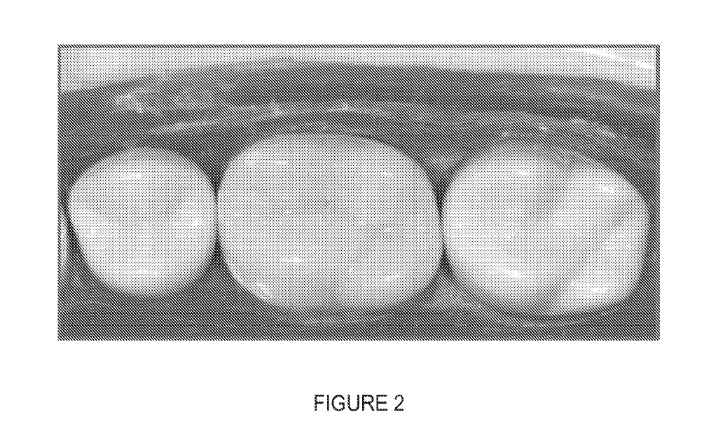

In another embodiment, teeth may further be attached and/or partially embedded in a surface, such as a dental arch model (FIG. 2), as may be appropriate for a given use; in this embodiment, the enamel layer may extend only to the surface of the dental arch (i.e.: the portion of the tooth embedded in the arch may lack the enamel layer).

The thermoset resin and the radio-opaque filler making up the dentin may the same or different that those in the enamel portion. In one embodiment, the thermoset resin and the radio-opaque filler making up the dentin are the same as those in the enamel portion. In one embodiment, the dentin thermoset resin comprises a polyurethane resin; in another embodiment, the radio-opaque filler comprises calcium carbonate.

The specific thermoset resin and filler to use in the enamel and/or dentin layers may vary depending on all factors to be considered for a particular intended use. Similarly, the ratio of resin to filler will vary depending on the specific resin/filler used. In one non-limiting embodiment, the filler is present in the enamel/dentin portions ranging from 0.1 g to 60 g per 30 cc of resin. In various further non-limiting embodiments, the filler is present in the enamel/dentin portions ranging from 0.1 g to 50 g, 0.1 g to 40 g, 0.1 g to 30 g, 0.2 g to 60 g, 0.2 g to 50 g, 0.2 g to 40 g, 0.2 g to 30 g, 0.4 g to 30 g, 0.8 g to 30 g, 1 g to 30 g, 2 g to 30 g, 4 g to 30 g, 5 g to 30 g, 10 g to 30 g, 20 g to 30 g, 0.1 g to 25 g, 0.1 g to 20 g, 0.1 g to 10 g, 0.1 g to 10 g, or 0.1 g to 5 g per 30 cc of resin. In a further non-limiting embodiment, when the thermoset resin comprises polyurethane and the filler comprises calcium carbonate, the calcium carbonate filler is present in the enamel/dentin portions ranging from 12 g to 28 g per 30 cc of polyurethane resin.

In a further non-limiting embodiment, the ratio of thermoset resin to radio-opaque filler material in the enamel portion is between about 1:1 to about 1.5:1 on a weight:volume basis. In this embodiment, the thermoset resin may comprise a polyurethane resin and the radio-opaque filler resin may comprise calcium carbonate. In various other embodiments, the ratio of thermoset resin to radio-opaque filler material in the enamel portion is between about 0.1 grams:30 cc to about 60 grams:30 cc weight:volume.

In another embodiment, the ratio of thermoset resin to radio-opaque filler material in the dentin portion is between about 1:1 to about 2:1 on a weight:volume basis. In this embodiment, the thermoset resin may comprise a polyurethane resin and the radio-opaque filler resin may comprise calcium carbonate. In various other embodiments, the ratio of thermoset resin to radio-opaque filler material in the dentin portion is between about 0.1 grams:30 cc to about 60 grams:30 cc weight:volume.

It may be useful to provide an artificial tooth with differential harness between the enamel portion and the dentin portion, to better simulate the hardness properties of natural teeth. Thus, in a further embodiment, the ratio of thermoset resin to radio-opaque filler material in the dentin portion is higher than the ratio of thermoset resin to radio-opaque filler material in the enamel portion. In this embodiment, where the enamel and the dentin portion have the same thermoset resin and filler, the enamel portion of the tooth will be harder than the dentin portion, which will better simulate the natural tooth. In one exemplary embodiment, the thermoset resin is polyurethane and the filler is calcium carbonate in both the enamel and dentin portions. As will be understood by those of skill in the art, the teeth may have additional layers (beside the enamel and dentin layers) that further differ in hardness.

In another embodiment, the dentin portion may comprise a different thermoset resin and/or filler that do not possess the same level of hardness as in the enamel portion.

In a further embodiment, the enamel portion and the dentin portion have a porosity of between about 0% to about 5%. Limiting the porosity within the artificial tooth helps to preserve appropriate strength characteristics. Any porosity present is evenly distributed throughout the mass of the enamel and/or dentin portions.

The enamel and dentin portions may be of any thickness suitable for an intended purpose. In one embodiment, the enamel and/or dentin portion thickness may be relatively uniform over the tooth length (for example, in a block embodiment). In another embodiment, the enamel and/or dentin portion thickness may vary over the length of the tooth, for example, as it does in naturally occurring teeth. In one non-limiting embodiment, the dentin portion is about 1.2× to about 6×, or about 1.5× to about 3× the thickness of the enamel portion. In one embodiment, the enamel portion thickness is between about 0.5 mm to about 3.4 mm in a gradient from the crown (thickest) to the root or surface in which the tooth is embedded (ex: dental arch model) (thinnest), and the dentin thickness is between about 3 mm and 9 mm in thickness (and relatively uniform over the length of the tooth. In another embodiment, the enamel:dentin ratio may vary from about 1:6 near the root or surface, to about 1:2.6 near the crown.

The artificial teeth of the present invention may comprise any other components desirable for an intended purpose, such as additional layers as noted above. In another embodiment, the artificial tooth further comprises a root portion/layer, wherein the crown is over or encompasses the root portion. The root portion can be made of any suitable material as deemed appropriate for an intended use. In various embodiments, the root structure may be made of materials including, but not limited to any plastic other than that used for the enamel and dentin portions/layers, such as nylon, ABS (Acrylonitrile butadiene styrene), PLA (polylactic acid), TPU (Thermoplastic polyurethane), HIPS (High impact Polystyrene), ABS (Acrylonitrile Butadiene Styrene), etc. The teeth may further comprise a root canal artifact, including but not limited to silver pins, wherein the crown is over or encompasses the root canal artifact.

In another embodiment, the artificial tooth further comprises a cavity. In one non-limiting embodiment, a defect may be introduced in the enamel to simulate a cavity. In another embodiment, the cavity is pre-manufactured and inserted into the mold while pouring the resin. The defect may be of any suitable size as appropriate to simulate a cavity; in one embodiment, the defect may penetrate through the enamel potion, exposing a region of the dentin portion. In another embodiment, the defect may extend to or through a segment of the dentin portion.

The artificial teeth of the present invention may be made by any suitable technique in light of the present disclosure; exemplary embodiments are provided in the examples that follow.

In another embodiment, a plurality of artificial teeth is disposed in an artificial dental arch model, to facilitate dental student training. In this embodiment, a bottom segment of the artificial teeth are affixed to the dental arch, with the crown portion exposed.

In a second aspect, the invention provides methods for making an artificial tooth, comprising:

(a) distributing a radio-opaque filler material within a thermoset resin to provide a modified resin; and

(b) molding the modified resin into an artificial tooth.

The methods of this second aspect of the invention can be used, for example, to make the artificial tooth of any embodiment or combination of embodiments of the first aspect of the invention. Any suitable molding techniques can be used, as will be understood by those of skill in the art in light of the present disclosure. Master molds may be cylinders, spheres, or any round surface to help maintain even coating of the mold surface, though this can also be done by vibration or shaking.

In one embodiment, the methods comprise producing layers of differing hardness; in one exemplary embodiment, the layers may comprise different polymers and/or different filler:polymer mix ratios as described herein. In another embodiment, molds can be made for rolling or vibrating and a pipet array can be used to flush the molds and coat the surfaces with traces amounts of resin/polymer, allowing density (hardness) gradients to be controlled.

Exemplary thermoset resins for the methods of the invention included but are not limited to polyester resins, epoxy resins, cyanate ester resins, vinyl cure resins, ultraviolet cure resins, BMI's (bismaleimides), polybenzoxazine, polyimide, phenolic resin, polyesters, polyurethanes, and others known to those skilled in the art. In one specific embodiment, the thermoset resin comprises a polyurethane resin. Exemplary radio-opaque fillers for use in the methods of the invention include, but are not limited to talc, silica, mica, calcium carbonate, barium sulfate, alumina, silica carbide, plastic monofilaments, carbon fiber, zirconia, borosilicate glass powder, radiopaque borosilicate powder, titanium dioxide, zinc oxide, and pigments. In one specific embodiment, the radio-opaque filler comprises calcium carbonate.

The filler may be distributed within the thermoset resin using any suitable technique. The filler may be treated in any suitable way to facilitate its distribution within the thermoset resin. In one embodiment, the filler may be treated to improve binding to the thermoset resin, including but not limited to silane coating of the filler. The filler is distributed within the thermoset resin, in that it is relatively homogenously dispersed throughout the resin and not clumped or aggregated.

In one embodiment, the radio-opaque filler material has an average size of between about 400 mesh and about 600 mesh, or between about 15-40 microns in diameter. As will be understood by those of skill in the art, a preferred average particle size depends on a number of factors, including but not limited to the thermoset resin in which the filler particle is to be distributed, the viscosity of the thermoset resin, etc.

The specific thermoset resin and filler may vary depending on all factors to be considered for a particular intended use. Similarly, the ratio of resin to filler will vary depending on the specific resin/filler used. In one non-limiting embodiment, the filler is added to the resin at ratios ranging from 0.1 g to 60 g per 30 cc of resin. In various further non-limiting embodiments, the filler is present in the enamel/dentin portions ranging from 0.1 g to 50 g, 0.1 g to 40 g, 0.1 g to 30 g, 0.2 g to 60 g, 0.2 g to 50 g, 0.2 g to 40 g, 0.2 g to 30 g, 0.4 g to 30 g, 0.8 g to 30 g, 1 g to 30 g, 2 g to 30 g, 4 g to 30 g, 5 g to 30 g, 10 g to 30 g, 20 g to 30 g, 0.1 g to 25 g, 0.1 g to 20 g, 0.1 g to 10 g, 0.1 g to 10 g, or 0.1 g to 5 g per 30 cc of resin. In a further non-limiting embodiment, when the thermoset resin comprises polyurethane and the filler comprises calcium carbonate, the calcium carbonate filler is added to the resin at ratios ranging from 12 g to 28 g per 30 cc of polyurethane resin.

Levels of 60 grams filler per 30 cc resin can be achieved by any suitable methods, including but not limited to molding methods comprising any permutation or combination of:

(1) desiccating the filler powder before mixing;

(2) vacuuming the molds after mixing; and

(3) vibrating the mold for the duration of cure time to limit settling of suspended filler particles.

In a further non-limiting embodiment, the ratio of thermoset resin to radio-opaque filler material is between about 1:1 to about 2:1 on a weight:volume basis. In this embodiment, the thermoset resin may comprise a polyurethane resin and the radio-opaque filler resin may comprise calcium carbonate.

### Example 1

### Radiopacity of Resin Formulations with Calcium Carbonate Filler

Plastic teeth have been traditionally used in dental education to ensure basic competency before students treat patients. Despite ubiquitous use in dental practice, procedures to bond resins to tooth structure cannot be mimicked with today's plastic teeth, and these teeth are virtually radiolucent.

Our goal is to incorporate calcium carbonate fillers to make resin teeth more etchable and radiopaque; this example focuses on radiopacity in the current work.

### Methods

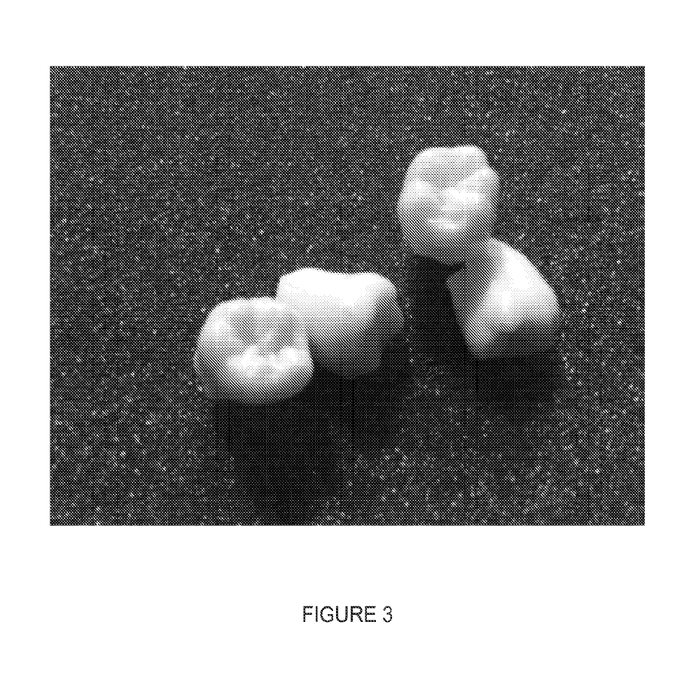

Two types of polyurethane resins—R1 (Smooth-Cast®325) and R2 (Task® 3), (Smooth-On Corp.) each with various CaCO₃ filler loads, were compared (Table 1). These resins possess different set times, surface tensions, and shrink rates (Task® 3: Compression Strength 8,300 psi, 0.0025 in/in shrinkage; Smooth-Cast®325: Compression Strength 3,500 psi, 0.01 in/in shrinkage). The resins were molded into an exact replica of a traditional plastic tooth (See FIG. 3). Specimens were radiographed using phosphor plates at 70 kVp, 0.32 seconds, and 7 mA, and a 300-mm focus film distance. Exposed radiographs were digitally scanned (MiPacs), and the average grey level of the coronal area (n=3) of each specimen was quantified using a software analysis program (ImageJ). The radiodensity was compared to a standard aluminum step wedge, a natural tooth and a traditional plastic tooth.

### Results

TABLE 1

| | Radiodensity | Aluminum Equivalent |
|---|---|---|
| Materials | (grey level) | Thickness (mm) |
| Enamel | 235.8 ± 7.3 | >10 |
| Dentin | 140.8 ± 7.2 | 5.6 |
| Traditional plastic tooth | 59.2 ± 1.5 | <1 |
| R1- No CaCO₃ | 51.1 ± 0.7 | <1 |
| R1- 30 g CaCO₃ | 115.2 ± 3.3 | 3.8 |
| R2- No CaCO₃ | 88.1 ± 1.5 | 2.4 |
| R2- 12 g CaCO₃ | 91.2 ± 1.4 | 2.6 |
| R2- 15 g CaCO₃ | 90.7 ± 2.9 | 2.5 |
| R2- 18 g CaCO₃ | 112.9 ± 2.8 | 3.6 |
| R2- 21 g CaCO₃ | 110.7 ± 2.9 | 3.4 |
| R2- 24 g CaCO₃ | 99.3 ± 2.0 | 3.1 |
| R2- 27 g CaCO₃ | 108.8 ± 0.7 | 3.4 |
| R2- 30 g CaCO₃ | 88.4 ± 1.6 | 2.6 |

### Conclusion

Addition of CaCO₃ improved the radiopacity of the resin formulations, with some approaching dentin radiopacity. Existing porosities in Task® 3 resins introduced variations in grey level readings.

### Example 2

### Hardness and Bondability of a Resin with Calcium Carbonate Filler

The goal of this experiment was to incorporate calcium carbonate fillers to make resin teeth used in pre-clinical dental education more etchable and radiopaque. We focused on determining the hardness and bondability of the new resin formulations in the current work.

### Methods

Two types of resins (Smooth-Cast® 325 vs. Task® 3), each with various CaCO₃ filler loads, were compared (Table 2). The resins were either molded into the exact replica of traditional plastic tooth or as rods. Human enamel was used as control.

### Hardness Test

A surface hardness test with the Rockwell Hardness Tester Scale Symbol A [Model 3JR, Wilson Mechanical Instrument Co] was used with a conical diamond indenter (Brale; 60 kg load) on resin rods (not tooth shaped for convenience and accuracy). The values are by Rockwell Hardness Number (RHN).

### Bond Strength Test

Shear bond strengths of composite to test teeth were conducted using a force gauge (DART Series Digital Force Gauge, Shimpo, Itasca, Ill.) on a motorized stand (Programmable Motorized Test Stand, Shimpo, Itasca, Ill.) to record the peak shear force (N). A cross-head speed of 5 mm/min was used.

The specimens were tooth shaped and prepared as follows. Composite resin (Filtek™ Supreme Plus, 3M ESPE) was injected into gelatin capsules and bonded perpendicularly to the axial surfaces of the samples. These areas had been previously prepared by flattening the surface, applying acid-etch for 15 s, and, finally, by applying D/E resin (All-Bond 2®, Bisco). Samples were light cured for 120 s, then tested 24 h later after storage in a water bath (37° C.).

Enamel was used as a control for both the hardness and bond strength tests. We also compared the experimental tooth forms to the currently used plastic tooth forms.

### Statistical Analysis

Groups with the same letter (Table) were not significantly different as determined using ANOVA with Tukey post-hoc analyses (α≦0.05).

### Results

TABLE 2

| | ROCKWELL | |
|---|---|---|
| | HARDNESS A TEST | BOND STRENGTH |
| MATERIALS | (RHN) | (N) |
| Control (Enamel) | >99 | 287.5 ± 70.1^{A} |
| Smooth-Cast ® 325: | 61.60 ± 1.43^{a} | 220.9 ± 29.8^{BC} |
| 30 g CaCO₃ | | |
| Task ® 3: 0 g CaCO₃ | 68.25 ± 1.87^{b} | 217.1 ± 54.2^{BC} |
| Task ® 3: 18 g CaCO₃ | 61.80 ± 0.92^{a} | 228.0 ± 44.9^{AC} |
| Task ® 3: 30 g CaCO₃ | 58.60 ± 0.97^{b} | 173.5 ± 27.7^{BC} |

### Conclusions

Hardness decreased as the amount of filler increased. This is thought to be due to a weakness in the bond between the filler particles and resin matrix, which may be remedied by treating the filler particles to increase binding to the resin (for example, silane coating).

Bond strengths increased as filler particles were added but dropped off with the highest amount of filler tested.

1. An artificial tooth for dental practice treatments, comprising
(a) an enamel portion, wherein the enamel portion comprises a thermoset resin, and wherein the enamel portion comprises at least a portion of an outer surface of the tooth; and
(b) a radio-opaque filler material distributed within the thermoset resin.

2. The artificial tooth of claim 1, further comprising a dentin portion, wherein the dentin portion is at least partially within the enamel portion, and wherein the enamel portion and the dentin portion together comprise a crown, wherein the dentin portion comprises a thermoset resin in which a radio-opaque filler material is distributed.

3. The artificial tooth of claim 2, wherein the thermoset resin and the radio-opaque filler making up the dentin are the same as those in the enamel portion.

4. The artificial tooth of claim 1, wherein the ratio of thermoset resin to radio-opaque filler material in the enamel portion is between about 1:1 to about 1.5:1 on a weight:volume basis.

5. The artificial tooth of claim 2, wherein the ratio of thermoset resin to radio-opaque filler material in the dentin portion is between about 1:1 to about 2:1 on a weight:volume basis.

6. The artificial tooth of claim 5, wherein the ratio of thermoset resin to radio-opaque filler material in the dentin portion is higher than the ratio of thermoset resin to radio-opaque filler material in the enamel portion.

7. The artificial tooth of claim 1, wherein the radio-opaque filler material has an average particle size of between about 15 microns and about 40 microns.

8. The artificial tooth of claim 1, wherein the enamel portion and the dentin portion have a porosity of between about 0% to about 5%.

9. The artificial tooth of claim 1, wherein the radio-opaque filler material comprises a silane coating.

10. The artificial tooth of claim 1, wherein the thermoset resin comprises polyurethane.

11. The artificial tooth of claim 1, wherein the radio-opaque filler material comprises calcium carbonate.

12. The artificial tooth of claim 2, further comprising a root portion, wherein the crown is over the root portion.

13. The artificial tooth of claim 1, further comprising a cavity.

14. A method for making an artificial tooth, comprising:
(a) distributing a radio-opaque filler material within a thermoset resin to provide a modified resin; and
(b) molding the modified resin into an artificial tooth
